(19) United States
(12) Patent Application Publication
(10) Pub. No.: US 2019/0262087 A1
(43) Pub. Date: Aug. 29, 2019

(54) DEVICE, ENDOSCOPE ROBOT DEVICE, AND MEDICAL ROBOT DEVICE, FOR DRIVING ROBOT JOINT USING WIRE

(71) Applicants: THE ASAN FOUNDATION, Seoul (KR); UNIVERSITY OF ULSAN FOUNDATION FOR INDUSTRY COOPERATION, Ulsan (KR)

(72) Inventors: Young Jin MOON, Gangdong-gu (KR); Jae Soon CHOI, Songpa-gu (KR); Hoyul LEE, Paju-si (KR)

(73) Assignees: THE ASAN FOUNDATION, Seoul (KR); UNIVERSITY OF ULSAN FOUNDATION FOR INDUSTRY COOPERATION, Ulsan (KR)

(21) Appl. No.: 16/413,022

(22) Filed: May 15, 2019

Related U.S. Application Data
(63) Continuation of application No. PCT/KR2017/011770, filed on Oct. 24, 2017.

(30) Foreign Application Priority Data
Nov. 16, 2016 (KR) 10-2016-0152825

(51) Int. Cl. A61B 34/30; B25J 9/10; B25J 9/06; A61B 34/00

(52) U.S. Cl. CPC A61B 34/30; B25J 9/104; A61B 2017/00314; A61B 34/71; A61B 2034/301; B25J 9/06

(57) ABSTRACT

Provided are a device for driving a robot joint using a wire, an endoscope robot device including the same, and a medical robot device including the same. The device for driving a robot joint includes at least: a base; a first link coupled with the base, and configured to be rotatable relative to the base; a second link coupled with the first link, and configured to be rotatable relative to the first link; a plurality of wires positioned opposite each other, and configured to generate motions of the first and second links; and a wire drive device configured to drive the plurality of wires. Each of the plurality of wires is fixed, via the second link, on one of the base and the first link. Positions where the plurality of wires are fixed are asymmetrical to each other and have different heights to each other.

### CROSS-REFERENCE TO RELATED APPLICATIONS

The present application is a continuation of International Patent Application No. PCT/KR2017/011770, filed Oct. 24, 2017, which claims the benefit of priority to Korean Patent Application No. 10-2016-0152825 filed on Nov. 16, 2016. The disclosures of the above-listed applications are hereby incorporated by reference herein in their entirety.

### RELATED ART

Embodiments of the inventive concept described herein relate to a device, an endoscopic robot device, and a medical robot device, for driving a robot joint using a wire.

Surgical robots or surgical instruments that are put into a human body have to be made compact, and hence a mechanism for driving a joint by a wire without directly mounting a motor to the joint is applied in many cases. Representative examples include commercial endoscopes, catheters, and surgical robots. A motor for driving a wire is provided inside a surgical robot. To increase the degree of freedom in motion of the surgical robot, the number of wires has to be increased. However, with the increase in the number of wires, the number of motors is also increased, and therefore it is difficult to make the surgical robot compact.

### SUMMARY

Embodiments of the inventive concept provide a device for driving a robot joint using a wire, the device being capable of generating various motions by a relatively small number of wires, and an endoscopic robot device and a medical robot device, which include the device.

The problems to be solved by the present disclosure are not limited to the aforementioned problems, and any other problems not mentioned herein will be clearly understood from the following description by those skilled in the art to which the present disclosure pertains.

According to exemplary embodiments, a device for driving a robot joint using a wire includes a base, a first link coupled with the base, and configured to be rotatable relative to the base, a second link coupled with the first link, and configured to be rotatable relative to the first link, a plurality of wires that are positioned opposite to each other and that generate motions of the first link and the second link, and a wire drive device that drives the plurality of wires. Each of the plurality of wires is fixed, via the second link, on one of the base and the first link, and positions where the plurality of wires are fixed are asymmetrical to each other and have different heights to each other.

According to other exemplary embodiments, a device for driving a robot joint using a wire includes a base, a robot arm including a plurality of links extending from the base and coupled with each other, and configured to be rotatable relative to each other, a plurality of wires positioned opposite to each other, and configured to generate a motion of the robot arm, and a wire drive device that drives the plurality of wires. Two or more of the plurality of wires are fixed, via a first link among the plurality of links, on one of the base and a second link among the plurality of links, and positions where the two or more of the plurality of wires are fixed are asymmetrical to each other and have different heights to each other.

According to other exemplary embodiments, a device for driving a robot joint using a wire includes a base, a robot arm including a plurality of links extending from the base and coupled with each other, and configured to be rotatable relative to each other, a plurality of wires positioned opposite to each other, and configured to generate a motion of the robot arm, and a wire drive device that drives the plurality of wires. The plurality of wires are fixed, via a first link among the plurality of links, to one of the base and a second link among the plurality of links, positions where the plurality of wires are fixed are symmetrical to each other. The device further includes springs interposed between the base and the first link and between the first link and the second link, respectively.

Other specific details of embodiments of the present disclosure are included in the detailed description and the drawings.

### BRIEF DESCRIPTION OF DRAWINGS

The above and other objects and features will become apparent from the following description with reference to the following figures, wherein like reference numerals refer to like parts throughout the various figures unless otherwise specified, and wherein:

FIG. 1 is a schematic view illustrating a configuration of a device for driving a robot joint using a wire according to some embodiments of the present disclosure;

FIG. 2 is a schematic view illustrating a drive mechanism of the device of FIG. 1 for driving the robot joint using the wire;

FIGS. 3A to 3C are schematic views illustrating motions of the device of FIG. 1 for driving the robot joint using the wire;

FIG. 4 is a schematic view illustrating a configuration of a device for driving a robot joint using a wire according to some embodiments of the present disclosure;

FIG. 5 is a schematic view illustrating a configuration of a device for driving a robot joint using a wire according to some embodiments of the present disclosure;

FIG. 6 is a schematic view illustrating a drive mechanism of the device of FIG. 5 for driving the robot joint using the wire;

FIG. 7 is a schematic view illustrating a motion of the device of FIG. 5 for driving the robot joint using the wire;

FIG. 8 is a schematic view illustrating a configuration of a device for driving a robot joint using a wire according to some embodiments of the present disclosure;

FIG. 9 is a schematic view illustrating a drive mechanism of the device of FIG. 8 for driving the robot joint using the wire;

FIG. 10 is a schematic view illustrating a motion of the device of FIG. 8 for driving the robot joint using the wire;

FIGS. 11A and 11B are schematic views illustrating a configuration of a device for driving a robot joint using a wire according to some embodiments of the present disclosure;

FIG. 12 is a schematic view illustrating an endoscopic robot device including a device for driving a robot joint using a wire according to some embodiments of the present disclosure; and

FIG. 13 is a schematic view illustrating a medical robot device including a device for driving a robot joint using a wire according to some embodiments of the present disclosure.

### DETAILED DESCRIPTION

The above and other aspects, features, and advantages of the inventive concept will become apparent from the following description of embodiments given in conjunction with the accompanying drawings. However, the inventive concept is not limited to the embodiments disclosed herein and may be implemented in various different forms. Herein, the embodiments are provided to provide complete disclosure of the inventive concept and to provide thorough understanding of the inventive concept to those skilled in the art to which the inventive concept pertains, and the scope of the inventive concept should be limited only by the accompanying claims and equivalents thereof.

Terms used herein are only for description of embodiments and are not intended to limit the inventive concept. As used herein, the singular forms are intended to include the plural forms as well, unless context clearly indicates otherwise. It will be further understood that the terms “comprise” and/or “comprising” specify the presence of stated features, components, and/or operations, but do not preclude the presence or addition of one or more other features, components, and/or operations. In addition, like numerals will denote like components throughout the specification, and the meaning of “and/or” includes each mentioned item and every combination of mentioned items. It will be understood that, although the terms first, second, etc. may be used herein to describe various components, these components should not be limited by these terms. These terms are only used to distinguish one component from another component. Thus, a first component discussed below could be termed a second component without departing from the teachings of the inventive concept.

Unless otherwise defined, all terms (including technical and scientific terms) used herein have the same meaning as commonly understood by those skilled in the art to which the inventive concept pertains. It will be further understood that terms, such as those defined in commonly used dictionaries, should not be interpreted in an idealized or overly formal sense unless expressly so defined herein.

Spatially relative terms, such as “beneath”, “below”, “lower”, “above”, “upper” and the like, may be used herein for ease of description to describe one component or feature's relationship to another component(s) or feature(s) as illustrated in the figures. It will be understood that the spatially relative terms are intended to encompass different orientations of the device in use or operation in addition to the orientation depicted in the figures. For example, if the device in the figures is turned over, components described as “below” or “beneath” other components or features would then be oriented “above” the other components or features. Thus, the exemplary term “below” can encompass both an orientation of above and below. The device may be otherwise oriented (rotated 90 degrees or at other orientations) and the spatially relative descriptors used herein interpreted accordingly.

Hereinafter, embodiments of the inventive concept will be described in detail with reference to the accompanying drawings.

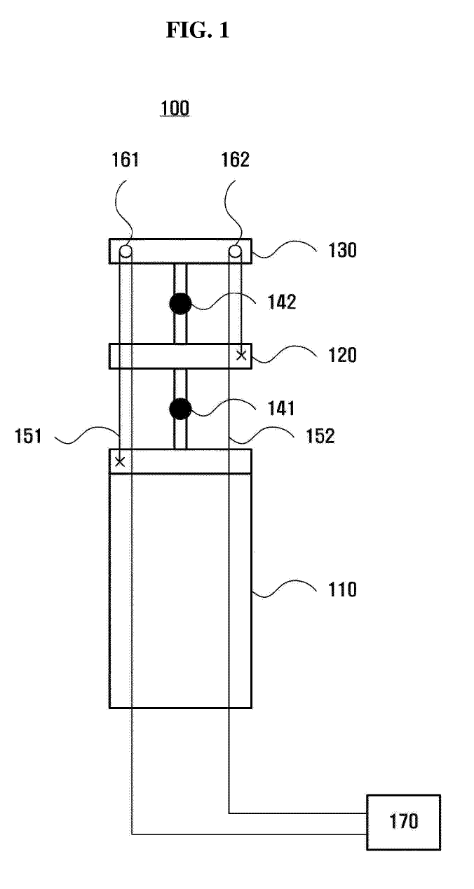

FIG. 1 is a schematic view illustrating a configuration of a device for driving a robot joint using a wire according to some embodiments of the present disclosure.

Referring to FIG. 1, the device 100 for driving the robot joint using the wire includes a base 110, a plurality of links 120 and 130, a pair of wires 151 and 152 for generating motions of the plurality of links 120 and 130, and a wire drive device 170 for driving the wires 151 and 152.

The plurality of links 120 and 130 are extended from the base 110 and are disposed adjacent to each other. The plurality of links 120 and 130 are coupled with the base 110 along with the same axial direction. The first link 120 is coupled with the base 110 through a rotary joint 141, and the second link 130 is coupled with the first link 120 through a rotary joint 142. In some embodiments, each of the links 120 and 130 has a ring shape. In some embodiments, each of the links 120 and 130 is coupled with one of the base 110 and the other link 120 or 130, through two or more rotary joints. Accordingly, the first link 120 is configured to be rotatable relative to the base 110, and the second link 130 is configured to be rotatable relative to the first link 120. In some embodiments, a plurality of other links (not shown in FIG. 1) are additionally interposed between the base 110 and the first link 120 or between the first link 120 and the second link 130. The plurality of links 120 and 130 configured to be rotatable relative to each other constitute at least part of a robot arm.

The pair of wires 151 and 152 are coupled to the wire drive device 170 at one end thereof and fixed on the plurality of links 120 and 130 at the other end thereof. The first wire 151 is extended to one side of the robot arm (e.g., the left side in FIG. 1) and is fixed, and the second wire 152 extends to an opposite side of the robot arm (e.g., the right side in FIG. 1) and is fixed. The pair of wires 151 and 152 are pulled or released by the wire drive device 170 to generate motions of the plurality of links 120 and 130. Accordingly, in some embodiments, the robot arm is bent toward the one side or the opposite side.

The wire drive device 170 drives the plurality of links 120 and 130 through the pair of wires 151 and 152. The wire drive device 170 pulls or releases the pair of wires 151 and 152 to cause the plurality of links 120 and 130 to generate motions while rotating. For example, the wire drive device 170 is implemented with at least one of a DC motor, a servo motor, and a step motor. However, the present disclosure is not limited thereto.

In the device 100 for driving the robot joint using the wire according to the some embodiments of the present disclosure, at least one of the pair of wires 151 and 152 is not directly fixed to the base 110, the first link 120, or the second link 130, but fixed thereto via at least one link disposed on an upper side of the fixed position, unlike in a conventional device for driving a robot joint using a wire. That is, in some embodiments, at least one of the pair of wires 151 and 152 is fixed, via one of the plurality of links 120 and 130, on the base 120 or the other one of the plurality of links 120 and 130. Furthermore, the pair of wires 151 and 152 positioned opposite to each other, and positions where the pair of wires 151 and 152 are fixed on the different links 120 and 130 are asymmetrical to each other and have different height to each other.

Referring to the some embodiments illustrated in FIG. 1, the first wire 151 is fixed, via the second link 130, on the base 110. A winding part 161 for winding the first wire 151 is provided on one side of the second link 130. The second wire 152 is fixed, via the second link 130, on the first link 120. A winding part 162 for winding the second wire 152 is provided on an opposite side of the second link 130. In some embodiments , the winding parts 161 and 162 are implemented with at least one of a pulley, a protrusion, and an opening. However, the present disclosure is not limited thereto.

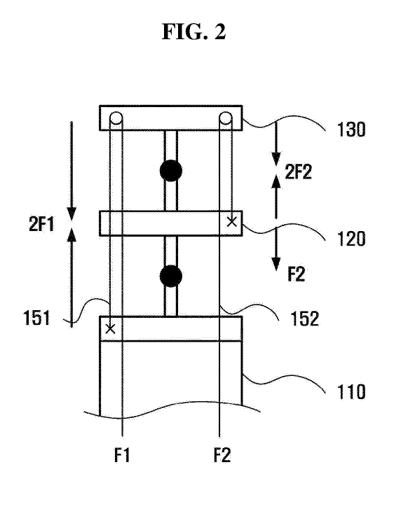

FIG. 2 is a schematic view illustrating a drive mechanism of the device of FIG. 1 for driving the robot joint using the wire.

Referring to FIG. 2, a movable pulley effect is applied to the robot because the pair of wires 151 and 152 are fixed on the base 110 and one link 120 via the other link 130 disposed on the upper side. Forces two times greater than forces pulling the wires from the outside are applied to the plurality of links 120 and 130 by the movable pulley effect. Furthermore, various motions are generated by the difference between the forces applied to the respective links because the pair of wires 151 and 152 positioned opposite to each other are asymmetrically fixed on the different links 120 and 130.

According to a conventional mechanism for driving a joint by a wire, differently from what is shown in FIG. 2, a wire fastening method in which one of a pair of wires is fixed to the one side of the second link 130 and the other wire is fixed to the opposite side of the second link 130 is applied.

In the above case according to the conventional mechanism, a force of F1 pulling the wire on the one side and a force of F2 pulling the wire on the opposite side are identically transmitted to the target link 130. Therefore, in the case where F1 is greater than F2, the plurality of links 120 and 130 are all rotated toward the one side such that the robot arm is bent toward the one side, and in the case where F2 is greater than F1, the plurality of links 120 and 130 are all rotated toward the opposite side such that the robot arm is bent toward the opposite side. That is, in the above case according to the conventional mechanism, the plurality of links 120 and 130 are moved in the same direction without being independently controlled. In the above case according to the conventional mechanism, to independently control motions of the plurality of links 120 and 130, a pair of wires have to be added and fixed on one side and an opposite side of the second link 120.

By contrast to the aforementioned conventional mechanism, according to the some embodiments of the present disclosure, the movable pulley effect is applied so that a force of 2F1, which is two times greater than the force F1, pulling the wire 151 on the one side is applied between the base 110 and the second link 130, a force of 2F2, which is two times greater than the force F2, pulling the wire 152 on the opposite side is applied between the first link 120 and the second link 130, and a force F2 of the same magnitude is applied between the base 110 and the first link 120. Accordingly, when the wires 151 and 152 are pulled by predetermined forces, various motions of the robot arm are generated by the difference between forces applied to one side and an opposite side of each of the plurality of links 120 and 130.

In addition, in some embodiments, the plurality of links 120 and 130 are independently controlled to move in different directions.

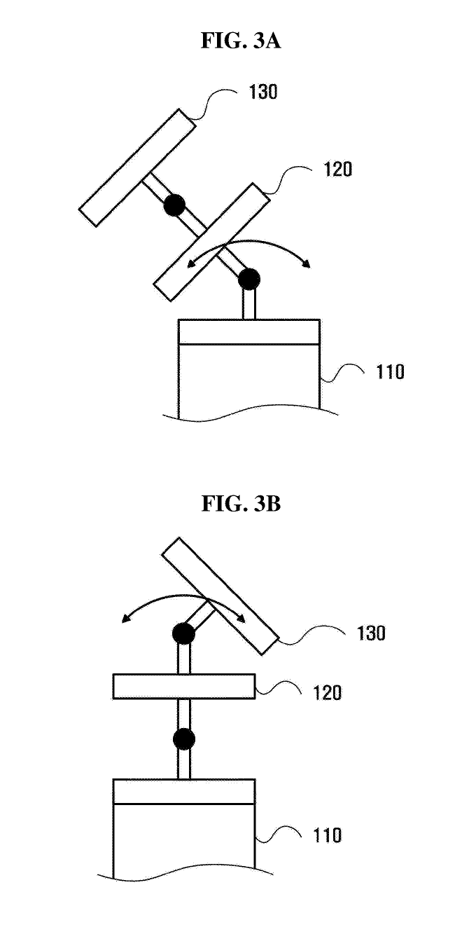

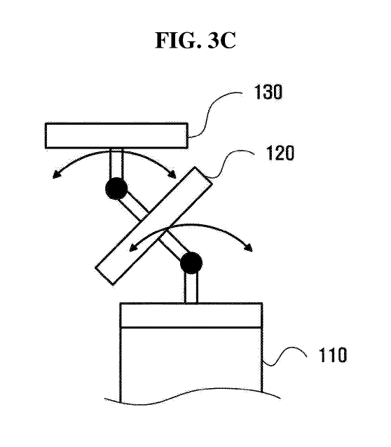

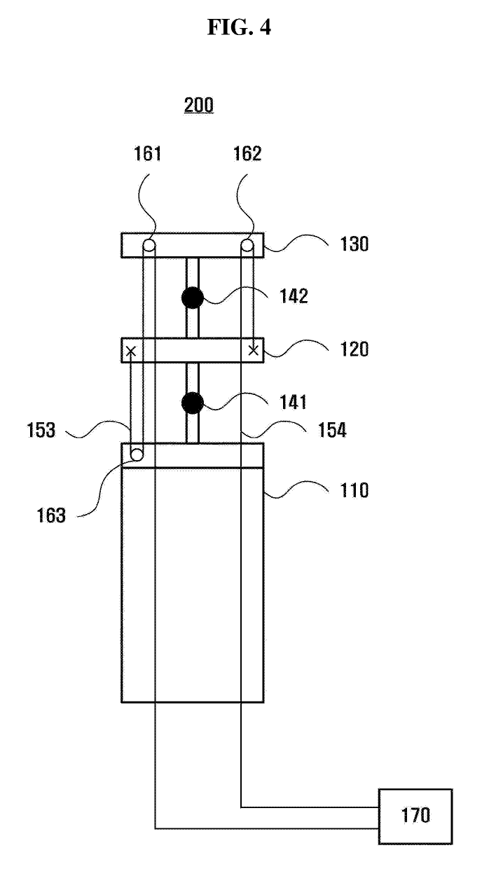

In some embodiments, the forces F1 and F2 pulling the pair of wires 151 and 152 are appropriately adjusted to bring both side forces applied between the first link 120 and the second link 130 into equilibrium and make one of both side forces applied between the base 110 and the first link 120 greater than the other. Accordingly, in some embodiments, only the first link 120 of the device 100 for driving the robot joint using the wire is rotated toward the one side or the opposite side such that the plurality of links 120 and 130 are integrally rotated, that is, only the lower part of the robot arm is bent toward the one side or the opposite side, as shown in FIG. 3A.

Alternatively, in some other embodiments, by bringing both the side forces applied between the base 110 and the first link 120 into equilibrium and making one of both the side forces applied between the first link 120 and the second link 130 greater than the other, only the second link 130 of the device 100 for driving the robot joint using the wire is rotated toward the one side or the opposite side, while the first link 120 fixed, as shown in FIG. 3B.

In some embodiments, the device 100 for driving the robot joint using the wire generates a motion in which both the first link 120 and the second link 130 are rotated. In such embodiments, the first link 120 and the second link 130 are independently rotated, and therefore, the first link 120 and the second link 130 are rotated in different directions. That is, for example, it is possible that the first link 120 is rotated toward the one side, and that the second link 130 is rotated toward the opposite side, as shown in FIG. 3C.

According to the some embodiments of the present disclosure, the motions of the robot arm for which two pairs of wires are required by the conventional mechanism for driving the joint by the wire is substantially identically implemented with only one pair of wires.

As the number of links constituting the robot arm increases, the number of wires and wire drive devices for controlling motions of the links also increases. Therefore, according to the conventional mechanism for driving the joint by the wire, which requires a number of wires, it is difficult to make the robot arm compact. By contrast, according to the some embodiments of the present disclosure, the device 100 generates substantially the same motions by using a smaller number of wires than the conventional mechanism for driving the joint by the wire, and thus the robot arm according to the present disclosure is made more compact.

FIG. 4 is a schematic view illustrating a configuration of a device for driving a robot joint using a wire according to some embodiments of the present disclosure. For convenience of description, the following description will be focused on the difference from FIG. 1.

Referring to FIG. 4, a pair of wires 153 and 154 of the device 200 for driving the robot joint using the wire are all fixed to the same link 120, unlike in the device 100 for driving the robot joint using the wire, which has been described above with reference to FIG. 1. The first wire 153 changes in direction twice. That is, the first wire 153 is fixed to a first link 120 sequentially via a second link 130 and a base 110. A winding part 161 for winding the first wire 153 is provided on one side of the second link 130, and a winding part 163 for winding the first wire 153 is provided on one side of the base 110. The second wire 154 is fixed to the first link 120 via the second link 130. A winding part 162 for winding the second wire 154 is provided on an opposite side of the second link 130, which is opposite to the one side for the winding part 161. In some embodiments, the winding parts 161, 162, and 163 are implemented with a pulley, a protrusion, and/or an opening. However, the present disclosure is not limited thereto.

A drive mechanism and motions of the device of FIG. 4 for driving the robot joint using the wire are substantially the same as those described above with reference to FIGS. 2 and 3A to 3C, differing only in the magnitudes of forces applied between the base 110 and the first link 120 and between the first link 120 and the second link 130 by a force pulling the wire 163 on the one side.

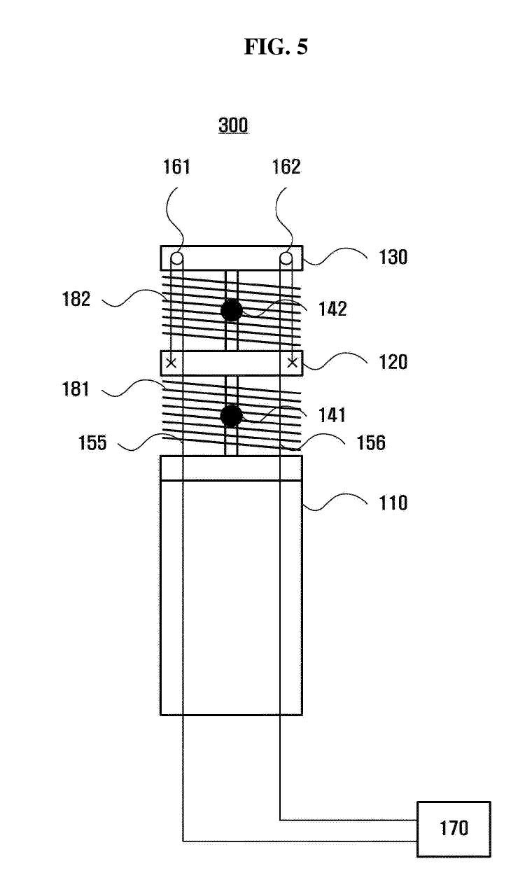

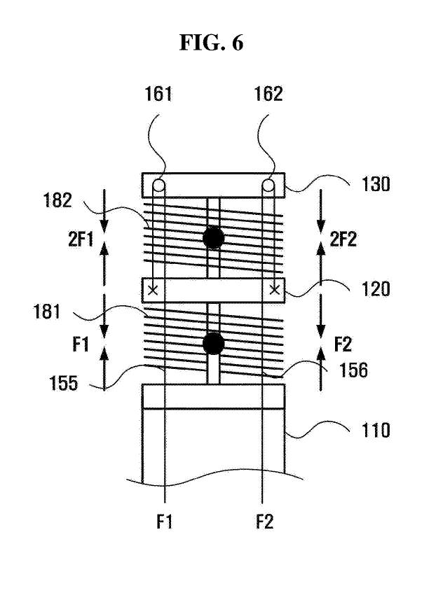

FIG. 5 is a schematic view illustrating a configuration of a device for driving a robot joint using a wire according to some embodiments of the present disclosure. For convenience of description, the following description will be focused on the difference from FIG. 1.

Referring to FIG. 5, a pair of wires 155 and 156 of the device 300 for driving the robot joint using the wire are all fixed to the same link 120, unlike in the device 100 for driving the robot joint using the wire, which has been described above with reference to FIG. 1. The pair of wires 155 and 156 are fixed to one side and an opposite side of the first link 120 via a second link 130, respectively. A winding part 161 for winding the wire 155 is provided on one side of the second link 130, and a winding part 162 for winding the wire 156 is provided on an opposite side of the second link 130. In some embodiments, the winding parts 161, 162, and 163 are implemented with a pulley, a protrusion, and/or an opening. However, the present disclosure is not limited thereto.

Additionally, springs 181 and 182 are interposed between the base 110 and the link 120 and between the link 120 and the link 130, respectively.

FIG. 6 is a schematic view illustrating a drive mechanism of the device of FIG. 5 for driving the robot joint using the wire.

Referring to FIG. 6, a movable pulley effect is applied so that a force of 2F1, which is two times greater than a force of F1, pulling the wire 155 on the one side is applied between the first link 120 and the second link 130 and a force F1 of the same magnitude is applied between the base 110 and the first link 120. Furthermore, a force of 2F2, which is two times greater than a force of F2, pulling the wire 156 on the opposite side is applied between the first link 120 and the second link 130, and a force F2 of the same magnitude is applied between the base 110 and the first link 120. Accordingly, when the wires 155 and 156 are pulled by predetermined forces, the magnitude of the force applied to the second link 130 is greater than the magnitude of the force applied to the first link 120.

Thus, in some embodiments, when the wires 155 and 156 are pulled, the second link 130 is rotated more than the first link 120. That is, in such embodiments, the upper part of the robot arm is bent more than the lower part thereof. As a result, in some embodiments, the robot arm has different curvatures (degrees of bending) for respective parts, rather than the same curvature as a whole. In some embodiments, the curvatures of the robot arm are adjusted depending on the number of wound wires 155 and 156 and/or the difference in stiffness between the springs 181 and 182.

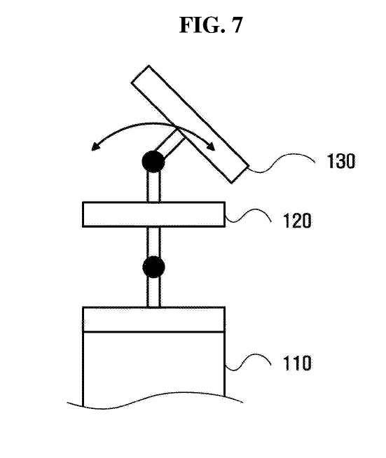

FIG. 7 is a schematic view illustrating a motion of the device of FIG. 5 for driving the robot joint using the wire.

According to the some embodiments of the present disclosure, the curvatures of the robot arm are changed or adjusted, which is impossible with the conventional mechanism for driving the joint by the wire.

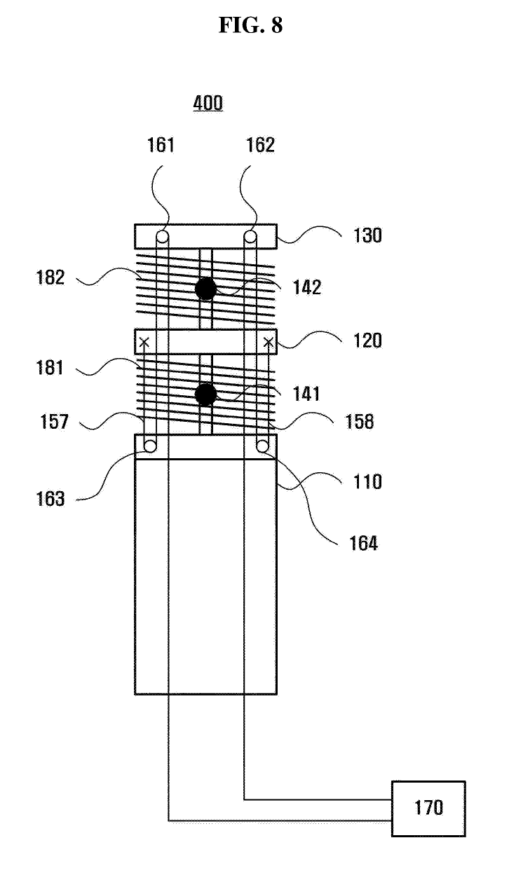

FIG. 8 is a schematic view illustrating a configuration of a device for driving a robot joint using a wire according to some embodiments of the present disclosure. For convenience of description, the following description will be focused on the difference from FIG. 1.

Referring to FIG. 8, a pair of wires 157 and 158 of the device 400 for driving the robot joint using the wire are all fixed to the same link 120, unlike in the device 100 for driving the robot joint using the wire, which has been described above with reference to FIG. 1. The pair of wires 157 and 158 all change in direction twice. That is, the pair of wires 157 and 158 are fixed to one side and an opposite side of the first link 120 sequentially via a second link 130 and a base 110. A winding part 161 for winding the wire 157 is provided on one side of the second link 130, and a winding part 162 for winding the wire 158 is provided on an opposite side of the second link 130. A winding part 163 for winding the wire 157 is provided on one side of the base 110, and a winding part 164 for winding the wire 158 is provided on an opposite side of the base 110. In some embodiments, the winding parts 161, 162, and 163 are implemented with a pulley, a protrusion, and/or an opening. However, the present disclosure is not limited thereto.

Additionally, springs 181 and 182 are interposed between the base 110 and the link 120 and between the link 120 and the link 130, respectively.

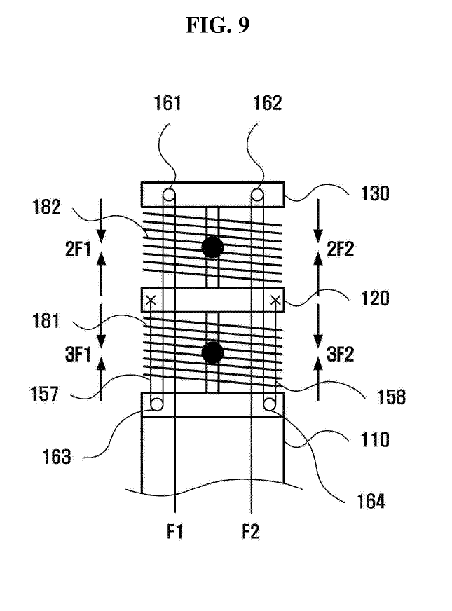

FIG. 9 is a schematic view illustrating a drive mechanism of the device of FIG. 8 for driving the robot joint using the wire.

Referring to FIG. 9, a movable pulley effect is applied so that a force of 2F1, which is two times greater than a force of F1, pulling the wire 157 on the one side is applied between the first link 120 and the second link 130 and a force of 3F1, which is three times greater than the force F1, is applied between the base 110 and the first link 120. Furthermore, a force of 2F2, which is two times greater than a force of F2, pulling the wire 158 on the opposite side is applied between the first link 120 and the second link 130, and a force of 3F2, which is three times greater than the force F2, is applied between the base 110 and the first link 120. Accordingly, when the wires 157 and 158 are pulled by predetermined forces, the magnitude of the force applied to the first link 120 is greater than the magnitude of the force applied to the second link 130.

Thus, in some embodiments, when the wires 157 and 158 are pulled, the first link 120 is rotated more than the second link 120. That is, in such embodiments, the lower part of the robot arm is bent more than the upper part thereof. As a result, in some embodiments, the robot arm has different curvatures (degrees of bending) for respective parts, rather than the same curvature as a whole. The curvatures of the robot arm are adjusted depending on the number of wound wires 157 and 158 and/or the difference in stiffness between the springs 181 and 182.

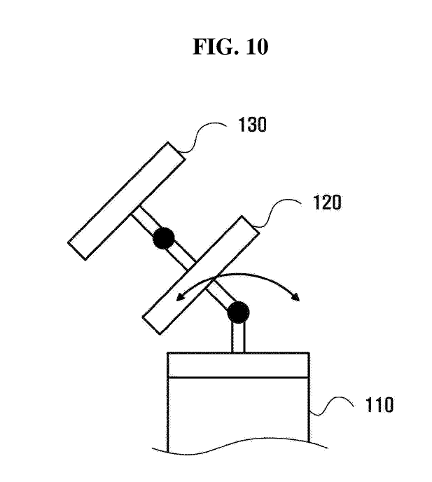

FIG. 10 is a schematic view illustrating a motion of the device of FIG. 8 for driving the robot joint using the wire.

Meanwhile, the wire fastening method is not limited to the some embodiments illustrated and may be variously modified in non-illustrated manners so as to be suitable for required motions according to the uses of the robot arm.

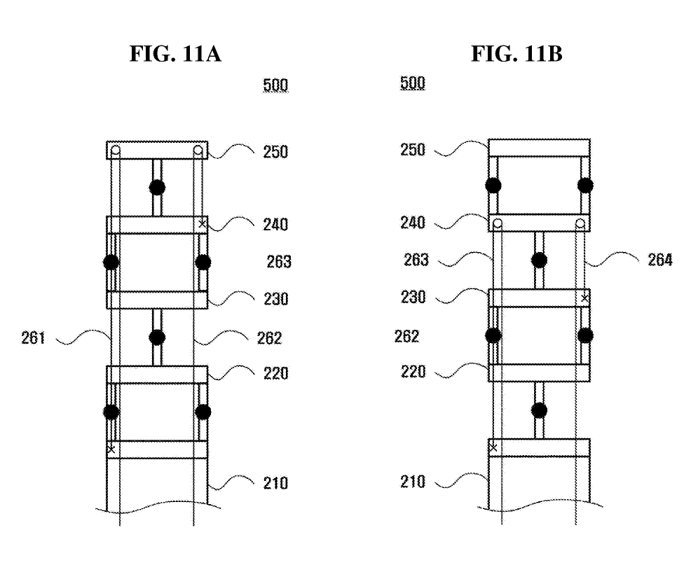

FIGS. 11A and 11B are schematic views illustrating a configuration of a device for driving a robot joint using a wire according to some embodiments of the present disclosure. FIG. 11A is a front view of the configuration of the device, and FIG. 11B is a side view of the configuration of the device.

Referring to FIGS. 11A and 11B, the device 500 for driving the robot joint using the wire includes a base 210, a plurality of links 220, 230, 240, and 250, two pairs of wires 261, 262, 263, and 264 for generating motions of the plurality of links 220, 230, 240, and 250, and a wire drive device (not illustrated) for driving the wires 261, 262, 263, and 264.

In some embodiments, each of the links 220, 230, 240, and 250 has a ring shape. The links 220, 230, 240, and 250 are alternately coupled together with respect to a vertical axis and a horizontal axis to increase a fastening force. That is, the first link 220 is coupled with the base 210 and configured to be rotatable in an up-down direction, and the second link 230 above the first link 220 is coupled with the first link 220 and configured to be rotatable in a left-right direction. In some embodiments, a plurality of other links are additionally interposed between the base 210 and the link 220 or between the links 220, 230, 240, and 250.

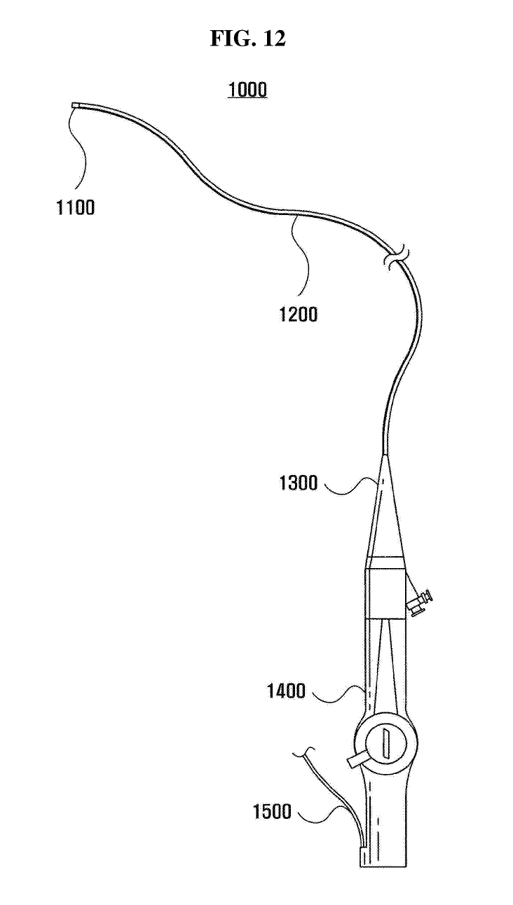

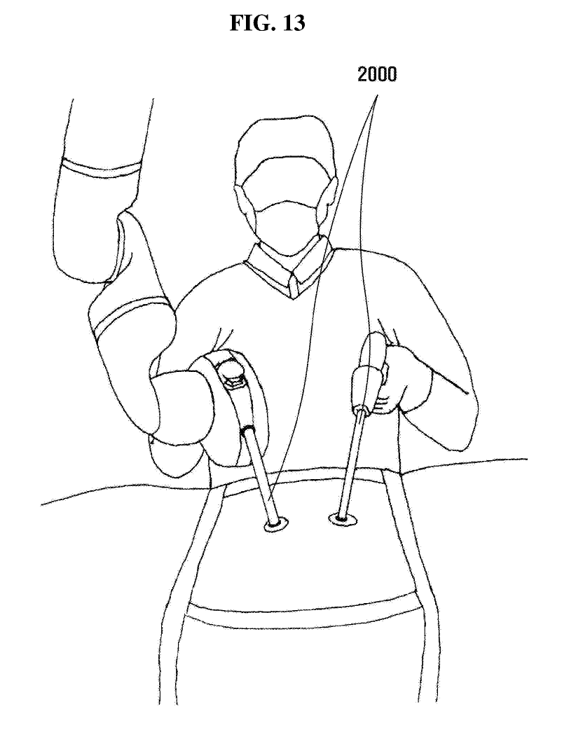

FIG. 12 is a schematic view illustrating an endoscopic robot device including a device for driving a robot joint using a wire according to some embodiments of the present disclosure.

Referring to FIG. 12, the endoscopic robot device 1000 includes a distal end part 1100, a shaft assembly 1200, a body assembly 1300, a control assembly 1400, and a connection assembly 1500.

The shaft assembly 1200 includes a bending section adjacent to the distal end part 110. In some embodiments, the bending section is bent according to a predetermined degree of freedom. In some embodiments, one or more the above-described devices 100, 200, 300, 400, and 500 for driving the robot joint using the wire according to the embodiments of the present disclosure are provided in the bending section of the shaft assembly 1200.

In some embodiments, the endoscopic robot device 1000 further include other components not explicitly illustrated in the present disclosure, based on knowledge of one of ordinary skill in the art.

FIG. 13 is a schematic view illustrating a medical robot device including a device for driving a robot joint using a wire according to some embodiments of the present disclosure.

Referring to FIG. 13, the medical robot device 2000 includes a robot arm (not illustrated), and the robot arm has an end effector on an end portion thereof, which has a predetermined function such as grip, incision, or claw.

In some embodiments, one or more of the above-described devices 100, 200, 300, 400, and 500 for driving the robot joint using the wire according to the embodiments of the present disclosure are provided in the robot arm or the end effector of the medical robot device 2000.

According to some embodiments of the present disclosure, the above-described devices 100, 200, 300, 400, and 500 for driving the robot joint using the wire is controlled by a computer processor that includes all the various devices capable of performing arithmetic processing. For example, the processor corresponds to not only a computer, such as a desktop personal computer (PC) or a notebook, but also a smart phone, a tablet PC, a cellular phone, a mobile terminal, and the like. Furthermore, instructions for controlling the above-described devices 100, 200, 300, 400, and 500 for driving the robot joint using the wire are stored in memory connected with the computer processor. In some embodiments, the above controlling is performed automatically according to the instructions. In some other embodiments, the above controlling is performed manually according to a user control.

According to the device for driving the robot joint using the wire according to the inventive concept, a movable pulley effect is applied to the links constituting the robot arm, and various motions are generated by a relatively small number of wires according to the difference between forces applied to the plurality of wires opposite to each other.

Effects of the inventive concept are not limited to the aforementioned effects, and any other effects not mentioned herein will be clearly understood from the following description by those skilled in the art to which the inventive concept pertains.

While the inventive concept has been described with reference to exemplary embodiments, it will be apparent to those skilled in the art that various changes and modifications may be made without departing from the spirit and scope of the inventive concept. Therefore, it should be understood that the above embodiments are not limiting, but illustrative.

What is claimed is:

1. A device for driving a robot joint using a wire, the device comprising:
a base;
a first link coupled with the base, and configured to be rotatable relative to the base;
a second link coupled with the first link, and configured to be rotatable relative to the first link;
a plurality of wires positioned opposite to each other, the plurality of wires being configured to generate motions of the first link and the second link; and
a wire drive device configured to drive the plurality of wires,
wherein each of the plurality of wires is fixed, via the second link, on one of the base and the first link, and positions where the plurality of wires are fixed are asymmetrical to each other and have different heights to each other.

2. The device of claim 1, wherein the second link includes at least one winding part around which the plurality of wires are wound.

3. The device of claim 2, wherein the at least one winding part includes at least one of a pulley, a protrusion, and an opening.

4. The device of claim 1, wherein at least one of the plurality of wires is fixed on the first link via the second link and further via the base.

5. The device of claim 4, wherein the base includes at least one winding part around which the at least one wire among the plurality of wires is wound.

6. The device of claim 1, further comprising:
springs interposed between the base and the first link and between the first link and the second link.

7. The device of claim 6, wherein the springs comprise
a first spring interposed between the base and the first link, and
a second spring interposed between the first link and the second link.

8. The device of claim 1, wherein
the plurality of wires comprise a first wire and a second wire,
the first wire is positioned opposite to the second wire,
the first wire is fixed, via the second link, on a first portion of the base,
the second wire is fixed, via the second link, on a second portion of the first link, and
the first portion of the base is positioned in a first height that is different from a second height of the second portion of the first link.

9. An endoscopic robot device comprising the device of claim 1 for driving the robot joint using the wire.

10. A medical robot device comprising the device of claim 1 for driving the robot joint using the wire.

11. A device for driving a robot joint using a wire, the device comprising:
a base;
a robot arm including a plurality of links, the plurality of links are extending from the base, coupled with each other, and configured to be rotatable relative to each other;
a plurality of wires positioned opposite to each other, the plurality of wires are configured to generate a motion of the robot arm; and
a wire drive device configured to drive the plurality of wires,
wherein two or more of the plurality of wires are fixed, via a first link among the plurality of links, on one of the base and a second link among the plurality of links, and positions where the two or more of the plurality of wires are fixed are asymmetrical to each other and have different heights to each other.

12. An endoscopic robot device comprising the device of claim 11 for driving the robot joint using the wire.

13. A medical robot device comprising the device of claim 11 for driving the robot joint using the wire.

14. A device for driving a robot joint using a wire, the device comprising:
a base;
a robot arm including a plurality of links, the plurality of links are extending from the base, coupled with each other, and configured to be rotatable relative to each other;
a plurality of wires positioned opposite to each other, the plurality of wires are configured to generate a motion of the robot arm; and
a wire drive device configured to drive the plurality of wires,
wherein the plurality of wires are fixed, via a first link among the plurality of links, on one of the base and a second link among the plurality of links, and positions where the plurality of wires are fixed are symmetrical to each other, and
wherein the device further comprises springs interposed between the base and the first link and between the first link and the second link.

15. The device of claim 14, wherein the springs comprise
a first spring interposed between the base and the first link, and
a second spring interposed between the first link and the second link.

16. The device of claim 14, wherein
the plurality of wires includes a first wire and a second wire,
the first wire is fixed, via the first line and via the base, on the second link,
the second wire is fixed, via only the first line, on the second link.

17. The device of claim 14, wherein
the plurality of wires includes a first wire and a second wire,
the first wire is fixed, via the first line and via the base, on the second link,
the second wire is fixed, via the first line and via the base, on the second link.

18. An endoscopic robot device comprising the device of claim 14 for driving the robot joint using the wire.

19. A medical robot device comprising the device of claim 14 for driving the robot joint using the wire.
